(19) United States
(12) Patent Application Publication
(10) Pub. No.: US 2019/0117243 A1
(43) Pub. Date: Apr. 25, 2019

(54) APPARATUS AND METHOD FOR IMPROVED CAVITATION-INDUCED DRUG DELIVERY

(71) Applicant: University of Washington, Seattle, WA (US)

(72) Inventors: Tatiana Khokhlova, Seattle, WA (US); Vera Khoklova, Seattle, WA (US); Oleg A. Sapozhnikov, Seattle, WA (US); Wayne Kreider, Seattle, WA (US); Adam D. Maxwell, Seattle, WA (US); Joo Ha Hwang, Seattle, WA (US)

(73) Assignee: University of Washington, Seattle, WA (US)

(21) Appl. No.: 16/169,945

(22) Filed: Oct. 24, 2018

Related U.S. Application Data
(60) Provisional application No. 62/576,490, filed on Oct. 24, 2017.

(51) Int. Cl. A61B 17/22; A61N 7/00

(52) U.S. Cl. CPC A61B 17/22004; A61N 7/00; A61H 23/008; A61N 2007/0052; A61N 2007/0039; A61B 2017/22008

(57) ABSTRACT

Apparatus and method for improved cavitation-induced drug delivery is disclosed. In one embodiment, a method for delivering a treatment composition to a target tissue using ultrasound includes: directing ultrasound waveforms toward the target tissue of a patient; generating ultrasound shock fronts at the target tissue of a patient; generating a cavitation inside the target tissue of a patient by the ultrasound shock front; and delivering the treatment composition to the patient. Absorption of the treatment composition by the target tissue is increased by the cavitation inside the target tissue. In some embodiments, the treatment composition may be delivered within a time period of +/−1 week from generating the cavitation.

### CROSS-REFERENCE(S) TO RELATED APPLICATION(S)

This application claims the benefit of Provisional Application No. 62/576,490, filed Oct. 24, 2017, which is incorporated herein by reference.

### STATEMENT OF GOVERNMENT LICENSE RIGHTS

This invention was made with government support under Grant Nos. R01EB023910, R01CA154451, R01EB015745 and R01EB007643 awarded by the National Institutes of Health. The government has certain rights in the invention.

### BACKGROUND

Drug delivery to a tumor or other solid malignancy is generally difficult because of increased interstitial pressure, high tumor cell density, and stromal barriers that inhibit drug delivery to the tumor. As a result, the therapeutic effects of intravascular nano-scaled drugs are limited by non-uniform trans-capillary transport and inhomogeneous interstitial transport. The transport barriers to drug delivery result from a dense interstitial structure (cellular of fibrous), abnormal blood and lymph vessel networks, elevated interstitial fluid pressure and interstitial contraction. These traits are shared across many malignancies, to varying extent, including those of the liver, pancreas, breast, brain, and prostate.

Some conventional technologies attempt to improve the delivery of drugs to the tumor through cavitation in the blood vessels. Such cavitation in the blood vessels may be induced by the ultrasound combined with systemically administered ultrasound contrast agents (UCA), which can take form of gas microbubbles that are artificially introduced into the blood flow. In some applications, the gas bubbles in the blood vessels, whether produced by ultrasound cavitation or being artificially introduced into the blood vessels, promotes the transport and distribution of the drugs at the target tumor. Generally, the UCAs help distribution of the drugs within the vasculature and toward the perivascular space. However, tumors are generally poorly vascularized, which limits drug delivery to the target regions of the tumor. Thus, even though the UCAs promote transportation of the drugs toward the tumor, the absorption of the drug by the tumor may remain weak, therefore limiting the effectiveness of the drug therapy. Accordingly, there remains a need for treatment systems that improve delivery of the drugs to the tumors and other solid malignancies.

### SUMMARY

This summary is provided to introduce a selection of concepts in a simplified form that are further described below in the Detailed Description. This summary is not intended to identify key features of the claimed subject matter.

Briefly, the inventive technology is directed to generating cavitation not only in blood vessels, but also in a tissue (e.g., a tumor). In operation, a pulsed focused ultrasound (pFUS) beam may be focused on the target tumor tissue to generate de-novo cavitation (as contrasted to artificially introduced gas bubbles or other ultrasound contrast agents). In some embodiments, the cavitation causes mechanical disruption of the target tissue (e.g., a stromal matrix), which in turn increases permeability of the target tissue to the medications (e.g., drug, chemotherapy, gene therapy, etc., collectively referred to as a “treatment composition”). In some embodiments, the absorption of the drug may be significantly increased. In some instances up to four-fold increase in uptake of a drug doxorubicin into the tumor was observed.

When the ultrasound is focused onto a region of tumor, a shock front develops within the focal waveform due to nonlinear propagation of the ultrasound toward the target tissue. In some embodiments, a peak negative pressures required to initiate (nucleate) and sustain inertial cavitation activity is relatively low (e.g., −2 to −10 MPa peak negative pressure), therefore being acceptable for clinical treatments. These relatively low negative pressures at the target tissue may be obtainable with a lens having a relatively high F-number, which is defined as a ratio of a focal length and a traverse size if the transducer. In some embodiments, a diagnostic probe with a high F-number may be reused as a therapy probe. In some embodiments, cavitation can be achieved using diagnostic ultrasound probes at relatively low mechanical index (MI) of 4-6.

In one embodiment, a method for delivering a treatment composition to a target tissue using ultrasound includes: directing ultrasound waveforms toward the target tissue of a patient; generating ultrasound shock waves at the target tissue of a patient; generating cavitation inside the target tissue of a patient by the ultrasound shock waves; and within a time period of +/−1 week from generating the cavitation, delivering the treatment composition to the patient, where an absorption of the treatment composition by the target tissue is increased by the cavitation inside the target tissue. In one aspect, the time period ranges from −1 hour to +48 hours. In another aspect, the ultrasound waveform is produced by an ultrasound transducer has an F-number within a 1-5 range. In one aspect, a characteristic dimension of the ultrasound transducer is less than 8 cm.

In one aspect, producing the ultrasound waveforms includes: producing a first burst of ultrasound waveforms within a first period of time, where the first period of time is shorter than 1 ms, and where the first burst of ultrasound waveforms is focused at a first segment of the target tissue; and producing a second burst of ultrasound waveforms within a second period of time, where the second period of time is shorter than 1 ms, where the second burst of ultrasound waveforms is focused at a second segment of the target tissue, and where the second segment is different than the first segment.

In one aspect, adjacent bursts of the ultrasound waveforms are separated by a rest time, wherein a ratio of a duration of the bursts and a duration of the rest times is a duty cycle of the treatment, and wherein the duty cycle of the treatment is less than 1%. In another aspect, a frequency of the ultrasound waveforms within the first burst and the second burst ranges from 0.5 MHz to 3 MHz. In one aspect, a burst-to-burst frequency is 1-200 Hz. In one aspect, the ultrasound shock waves inside the target tissue have a peak negative pressure within a range of −2 MPa to −10 MPa. In one aspect, the ultrasound shock waves inside the target tissue have a peak positive pressure within a range of 10 MPa to 70 MPa.

In one aspect, the treatment composition includes a chemotherapy treatment composition. In another aspect, the treatment composition includes a gene therapy. In one aspect, the target tissue comprises a tumor.

In one aspect, the treatment composition is administered before generating the cavitation, but not after generating the cavitation. In another aspect, the treatment composition is administered after generating the cavitation, but not before generating cavitation.

In one embodiment, a system for delivering a treatment composition to a target tissue using ultrasound includes: an ultrasound transducer configured for directing ultrasound waveforms toward the target tissue of a patient, where the initially smooth (e.g. sinusoidal or otherwise continuous) ultrasound waves transform to ultrasound shock waves at the target tissue of a patient, and where the ultrasound shock waves generate cavitation inside the target tissue. The treatment composition is delivered within a time period of +/−1 week from generating the cavitation, and an absorption of the treatment composition by the target tissue is increased by the cavitation inside the target tissue.

In one aspect, the system also includes a lens attached to the ultrasound transducer, where the lens has an F-number within a 1-5 range. In another aspect, a characteristic dimension of the ultrasound transducer is less than 8 cm.

In one aspect, a frequency of the ultrasound waveforms within the first burst and the second burst ranges from 0.5 MHz to 3 MHz. In another aspect, a burst-to-burst frequency is 1-200 Hz. In another aspect, the ultrasound shock waves inside the target tissue have a peak negative pressure within a range of −2 MPa to −10 MPa, and a peak positive pressure within a range of 10 MPa to 70 MPa.

### DESCRIPTION OF THE DRAWINGS

The foregoing aspects and many of the attendant advantages of the inventive technology will become more readily appreciated as the same are understood with reference to the following detailed description, when taken in conjunction with the accompanying drawings, wherein:

FIG. 1 is a partially schematic view of ultrasound system in accordance with an embodiment of the present technology;

FIG. 2 is an isometric view of a phased array ultrasound transducer in accordance with an embodiment of the present technology;

FIG. 3 is a partially schematic cross-sectional view of an ultrasound transducer in accordance with an embodiment of the present technology;

FIG. 4 is a photo of three ultrasound transducers in accordance with an embodiment of the present technology;

FIGS. 5A and 5B are graphs of pressure waveforms obtained with the phased array transducers in accordance with embodiments of the present technology;

FIG. 6 is an plan view of an object subjected to ultrasound cavitation in accordance with an embodiment of the present technology;

FIGS. 7A and 7B are graphs of spectral filter and ultrasound signal, respectively, obtained in accordance with embodiments of the present technology;

FIG. 7C is a schematic view of cavitation spots in accordance with an embodiment of the present technology;

FIGS. 8A and 8B are graphs of probability and persistence, respectively, vs. peak negative pressure of cavitation bubble formation in accordance with embodiments of the present technology;

FIG. 8C is a graph of ultrasound noise vs. peak negative pressure in accordance with an embodiment of the present technology;

FIGS. 9A-9C are graphs of pressure waveform parameters illustrating nonlinear distortion and shock formation in accordance with embodiments of the present technology; and

FIGS. 10A and 10B are graphs of one cycle of acoustic pressure and gas content of the cavitation bubbles, respectively, in accordance with embodiments of the present technology.

### DETAILED DESCRIPTION

While several embodiments have been illustrated and described, it will be appreciated that various changes can be made therein without departing from the spirit and scope of the claimed subject matter.

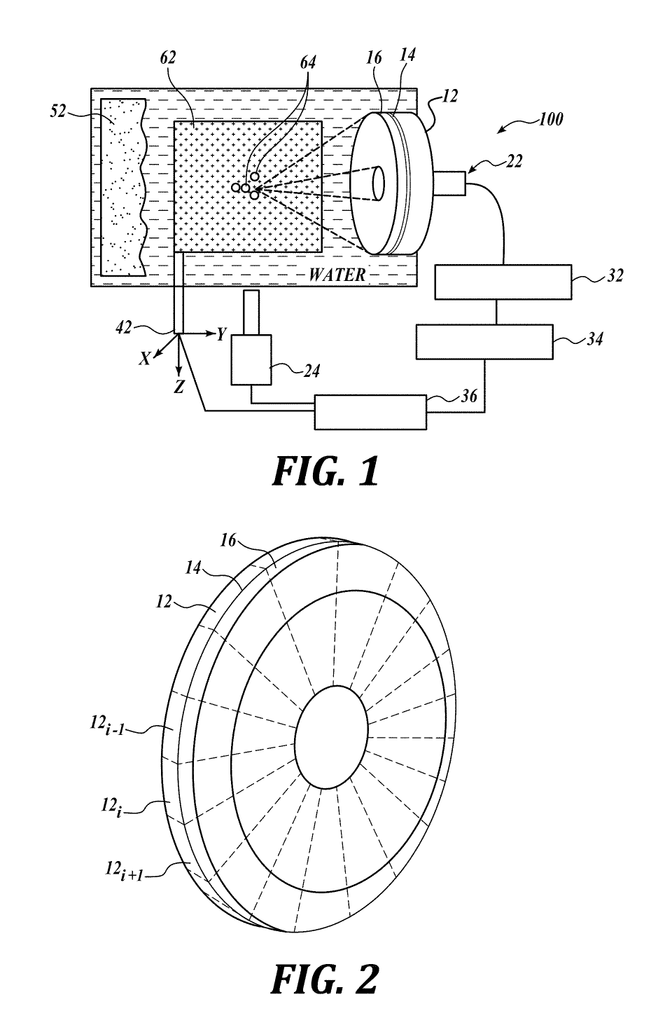

FIG. 1 is a partially schematic view of ultrasound system in accordance with an embodiment of the present technology. FIG. 1 illustrates an ultrasound system 100 having an ultrasound transducer 12, an interface 14 and a lens 16. In some embodiments, the ultrasound system 100 may operate without the lens 16. For example, the ultrasound may be focused onto a target by the shaped surface of the ultrasound transducer 12.

The transducer 12 can be a piezoelectric element that expands and shrinks with changing polarity of electrical voltage applied to the transducer. Such a change in electrical polarity can be applied by an alternating current (AC) at a target ultrasound frequency. In operation, the ultrasound transducer 12 vibrates at a prescribed frequency of a target ultrasound. (e.g., from about 20 kHz to about 10 MHz, from about 500 kHz to about 3 MHz, etc.). The interface 14 permanently attaches a lens to the transducer 12. The interface 14 is typically a permanent epoxy or other suitable strong adhesive.

In operation, the lens 16 focuses the ultrasound waveforms generated by the transducer 12 onto a target 62. As explained above, in different embodiments, the focusing may be provided by the curvature of the transducer only or by electronic phasing of a multi-element transducer elements. The target 62 may be a tumor, other tissue, an artificial laboratory target (e.g., a gypsum target or tissue phantom gel target, etc.). Ultrasound waveforms may travel through water that simulates a body under a treatment. The illustrated setup may include an ultrasound absorber 52 that limits reflection and scattering of the ultrasound into the environment. In some embodiments, the target may be mounted onto a three-dimensional (3D) positioning stage 42.

In operation, the ultrasound system directs ultrasound waveforms toward the target 62. The emitted ultrasound waveforms may start as smooth (harmonic) waveforms, and may develop into waveforms with shock fronts at the target location (focal area) of the target 62. These shock fronts generate cavitation bubbles 64 inside the target 62 (e.g., a tumor). After the cavitation bubbles 64 are formed at one location within the target 62 ultrasound, the field radiated by the system 100 can be redirected to another location within the target 62 (or, equivalently, the target 62 can be repositioned by the positioning stage 42) to create cavitation bubbles at the next location.

In some embodiments, the induction of cavitation throughout the tumor enhances the permeability of the tissue of the tumor, which in turn improves penetration of a pre- or post-administered treatment composition (e.g., drug, chemotherapy, gene therapy, etc.). The induction of cavitation may be performed immediately prior or after the administration of the treatment composition (e.g, within +/−1 week, within +/−48 hours; within+/−24 hours; within 1 hour prior and 24 hours after; within 1 hour prior and 12 hours after the administration of the treatment composition). Generally, no sedation or administrational ultrasound contrast agents are needed while seeding de-novo bubbles inside the target 62. Because of the enhanced permeability of the tumor tissue, in some embodiments a 4-5 fold increase in the delivery of the treatment composition can be achieved.

In some embodiments, a passive cavitation detector (PCD) or another ultrasound detector 22 can be used to detect and measure the activity of the cavitation bubbles 64 (whether targeting a phantom gel target or a patient body). The signals of the passive cavitation detector 22 may be amplified by a pre-amplifier 32 and may be interpreted by an oscilloscope or a signal analyzer 34. In some embodiments, an optical camera 24 (e.g., a high-speed camera) may be used to track the cavitation bubbles 64. The operation of the ultrasound system 100 may be controlled by a computer or other controller 36.

FIG. 2 is an isometric view of a phased array ultrasound transducer in accordance with an embodiment of the present technology. The illustrated phased array transducer includes an array of segments (e.g., transducer segments 12ᵢ₋₁, 12ᵢ, 12ᵢ₊₁, etc.) that can be individually activated at a prescribed phase offset. When the phase offsets among individual segments of the transducer 12 are properly accounted for, the individual activations of the segments of the transducer 12 result in the summations and cancellations of the ultrasound waveforms at the target. These summations and cancellations may result in improved targeting at the tumor or other target 62. For example, in some embodiments additional target focal areas may be possible by applying new sequences of the phase-offset activation of the transducer segments, without physically repositioning the phased array transducer. The segments of phased array transducer 12 may be connected to the lens 16 through the interface 14 (e.g., an epoxy).

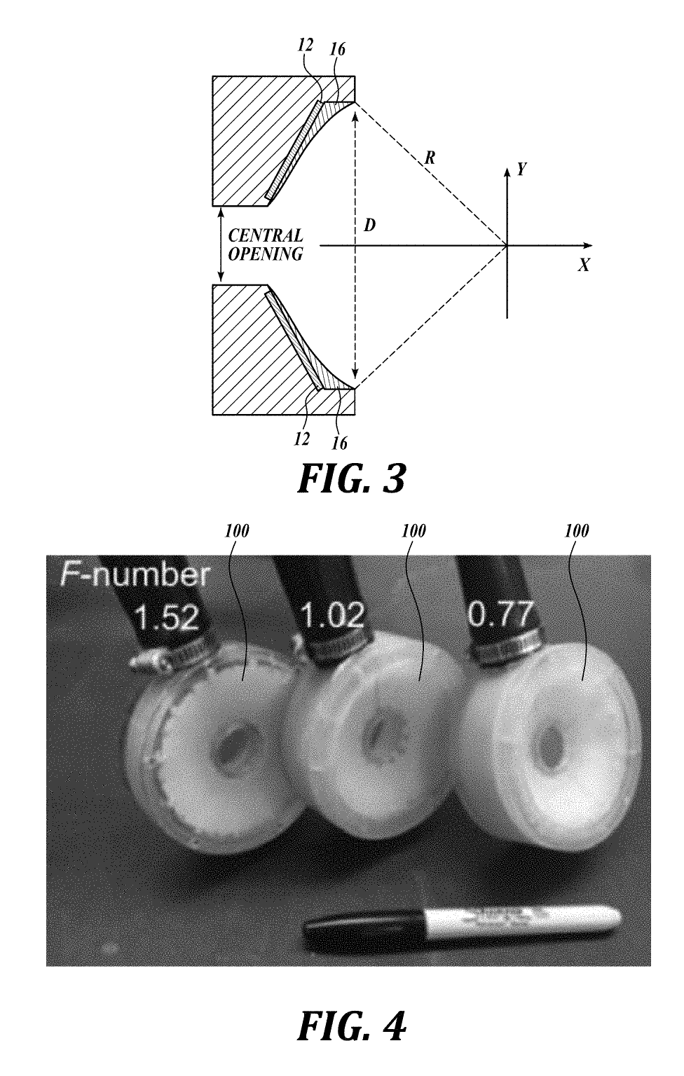

FIG. 3 is a partially schematic cross-sectional view of an ultrasound transducer in accordance with an embodiment of the present technology. In operation, the vibrations of the transducer 12 generate the ultrasound waveforms that are focused at the target by the lens 16. The transducer 12 and the lens 16 may include a central opening that houses a cavitation detector, for example, a passive cavitation detector.

In the illustrated embodiment, the lens 16 focuses ultrasound waveforms at the origin of the coordinate system x-y. Designation “R” represents focal distance of the lens 16, and designation “D” represents aperture of the lens. A focal number (F# or F-number) can be defined as the ratio R/D. In some embodiments of the present technology, the F-number ranges from about 0.75 to about 5, or from about 0.75 to about 1.5, but other ranges are also possible. Generally, the above-listed ranges of the F-number are considered relatively high (e.g., F-number>1), resulting in a relatively weak concentration of the ultrasound at the target area. The lenses having relatively high F-number may be beneficial because they require a smaller acoustic window to deliver the treatment and are therefore more practical clinically. Some examples of the lenses having various F-numbers are shown in FIG. 4.

FIG. 4 is a photo of the ultrasound transducers in accordance with an embodiment of the present technology. The illustrated transducers were attached to the corresponding lenses with an epoxy to form the ultrasound systems 100. In some embodiments, the transducers can be fabricated using flat, trapezoidal piezoelectric elements bonded with an adhesive acoustic matching layer to a matching rapid-prototyped plastic lens. The transducers had the optical aperture D of 73 mm, 75 mm, and 78 mm, respectively. The central opening was set uniformly at 20 mm diameter to allow for insertion of an in-line passive cavitation detector. The focal distances R were different: 56 mm, 76.6 mm, and 118 mm, respectively, resulting in F-numbers of 0.77, 1.02, and 1.52, respectively. In some embodiments, multiple transducers having different F-numbers may be used for a given treatment.

In some embodiments, the transducers are powered by a custom-built class D amplifier that is capable of delivering up to 26 kW pulse-average electrical power in pulses lasting up to 10 ms. The input waveforms to the amplifier may be generated by a computer-controlled field-programmable gate array (FPGA) board, but other sources of signal are also possible. As can be seen from the comparative size of a permanent marker at the bottom of FIG. 4, the illustrated ultrasound transducers are relatively small and suitable for application that treat tumors in humans or animals.

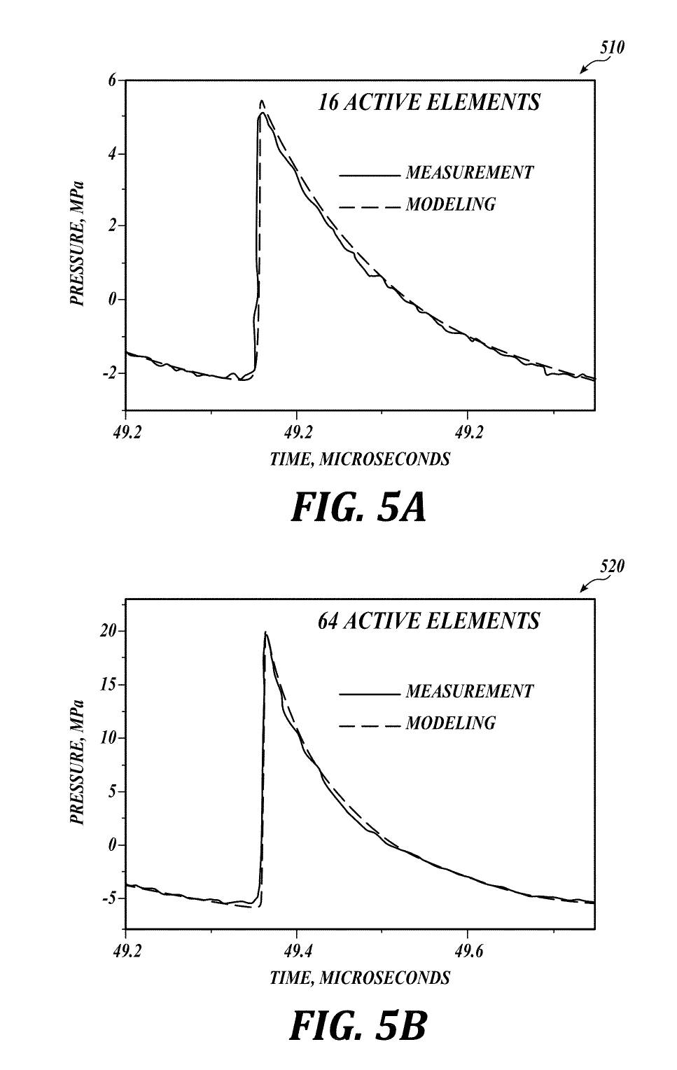

FIGS. 5A and 5B are graphs of pressure waveform obtained with the phased array transducer used for conventional ultrasound imaging in accordance with embodiments of the present technology. The horizontal axes in both graphs represent the time in microseconds counted from the moment of the transducer excitation. The vertical axes represent acoustic pressure at the target area (e.g., a region of tumor). The graphs include both measurement and modeling results. The graph in FIG. 5A corresponds to the phased array transducer with 16 active elements, and the graph in FIG. 5B corresponds to the phased array transducer with 64 active elements.

In both graphs, the ultrasound waveforms are significantly asymmetric and exhibit a shock front, even though the waveforms started as smooths functions at the ultrasound transducer. However, due to the nonlinear interactions along their propagation path, the waveforms became asymmetric and formed a shock front at the target.

In the illustrated embodiment, the peak negative pressures are relatively low: about −2.3 MPa in the graph of FIG. 5A, and about −5.5 MPa in the graph of FIG. 5B. In many applications, these relatively low peak negative pressures still result in cavitation at the target tissue, while limiting damage to the tissue.

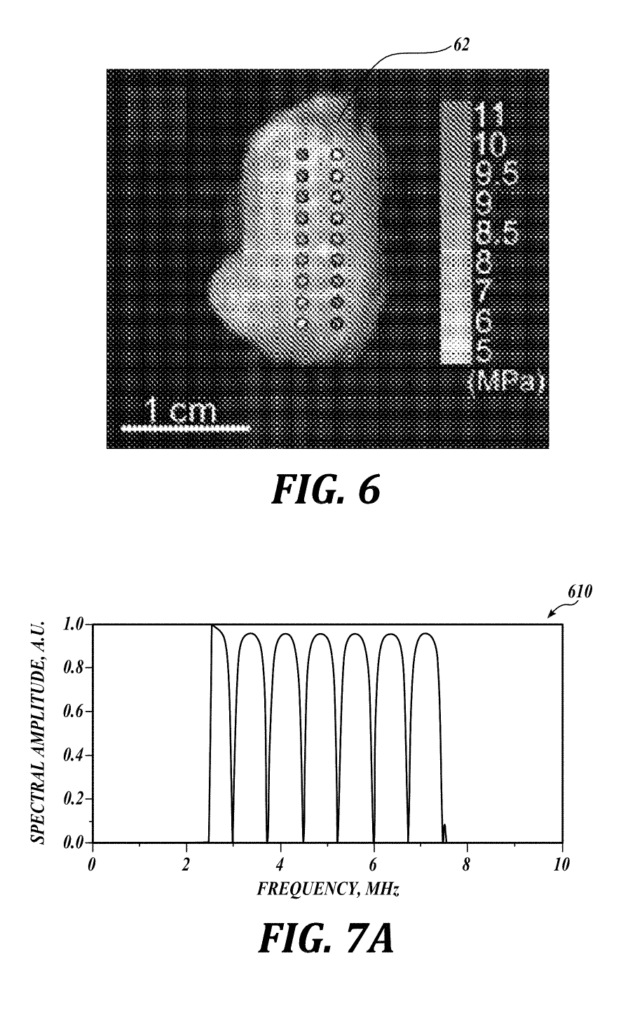

FIG. 6 is plan view of an object subjected to ultrasound cavitation in accordance with an embodiment of the present technology. The illustrated target corresponds to a mouse pancreatic tumor, observed with a fluorescent imaging system. In different embodiments, the target may be a tissue of a human patient. The target is subjected to a series of focused ultrasound pulses at different locations. In some embodiments, a transducer having an F-number of 1 emits ultrasound waveforms at 1 MHz. In the illustrated embodiment, the cavitation was triggered at 18 targets distributed over 9 rows and 2 columns. The illustrated ultrasound pulses have the peak negative pressures ranging from −5 MPa to −11 MPa, but other ranges are also possible. In some embodiments, the absorption of the drugs into the mouse pancreatic tumor was improved manifold due to increased permeability of the tissue. An added benefit of the inventive technology is that by focusing ultrasound onto the targeted tumor, the collateral damage to tissues outside of the target is avoided.

In different embodiments, different ultrasound parameters may be used for the pulsed focused ultrasound (pFUS) beam. Some representative, non-limiting examples of the pFUS parameters are:

F-number (F#=f/D): 1-5;

Transducer size: up to 8 cm diameter; up to 12 cm diameter;

Ultrasound frequency (within a burst): 0.5-3 MHz; 0.8-1.5 MHz;

Number of bursts of ultrasound per target location: 2-60; 1-100 (then move to the next target);

Burst-to-burst frequency (i.e., burst repetition frequency): 1-200 Hz;

Burst duration: 10 μs-1 ms;

Duty cycle: less than 1%, less than 2%;

Ultrasound peak positive pressure: 20-80 MPa; 10-70 MPa; 10-90 MPa;

Ultrasound peak negative pressure: −2 to −10 MPa; −2 to −5 MPa; Ultrasound treatment duration: 10-30 minutes; under 60 minutes;

Time window for treatment compound delivery: +/−1 day from ultrasound treatment; +/−2 days from ultrasound treatment; +/−1 week from ultrasound treatment; 1 hour before up to 24 hours after ultrasound treatment.

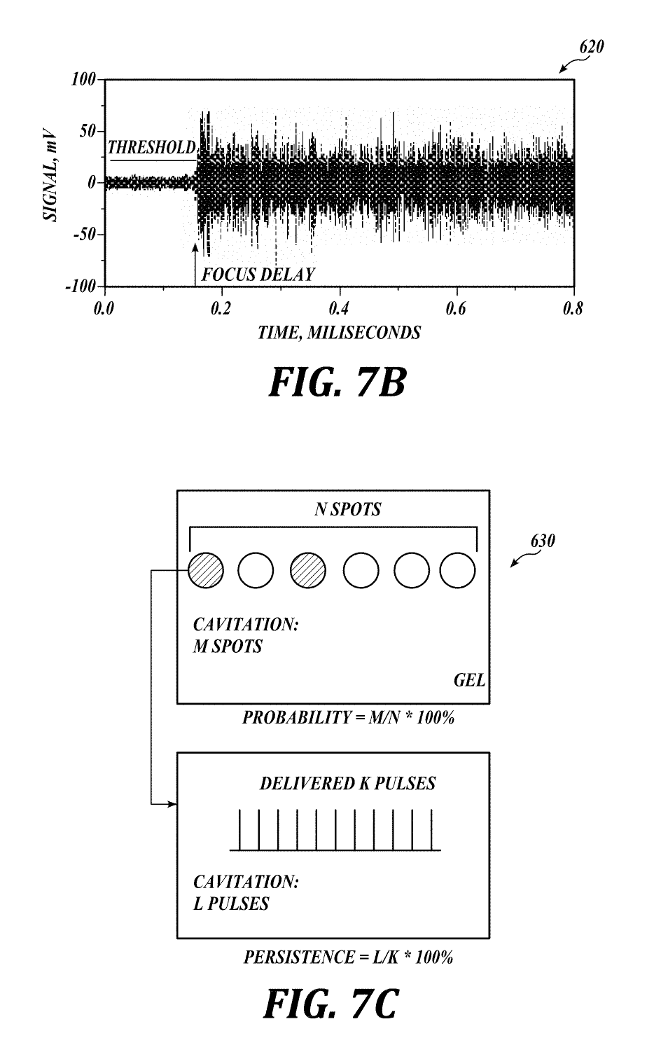

FIGS. 7A and 7B are graphs of the spectral filter and of the filtered ultrasound signal from the cavitation bubbles, respectively, obtained in accordance with embodiments of the present technology. The ultrasound signals may be acquired by, for example, passive cavitation detector 22. In the illustrated embodiments, a pulsed focused ultrasound (pFUS) beam had pulse duration of 1 ms, pulse repetition frequency (PRF) of 1 Hz, and overall duration of 60 seconds (i.e. 60 pulses delivered within 1 minute treatment time). Within each pulse, an ultrasound waveform (e.g. 1.5-5 MHz ultrasound tone burst) was produced by a single-element transducer. The F-number for different single-element transducers ranged from 0.75 to 1.5. The cavitation detector 22 acquired signals from the cavitation events. The acquired signals were processed as explained below.

FIG. 7A illustrates a frequency filter. The horizontal axis in FIG. 7A represents the filter frequency in MHz, and the vertical axis represents transmission coefficient of the filter. In general, the incoming signal corresponds to the activity of the cavitation bubbles generated at the target area. This incoming signal may be frequency-filtered using the filter shown in FIG. 7A, which is a combination of a band-pass filter (2.5-7.5 MHz) and a notch-shaped filter. As a result, the pulsed high intensity focused (HIFU) harmonics backscattered by the target tissue in the frequency domain are suppressed. The resulting filtered signal in time domain is shown in FIG. 7B.

FIG. 7B illustrates a filtered PCD signal in time domain. The horizontal axis in FIG. 7B represents time in milliseconds, and the vertical axis represents signal amplitude in mV. Here, a cavitation event is considered observed if the signal is larger than the noise by a factor of sqrt(5) (also referred to as the Rose criterion). This criterion (“threshold”) is represented by a horizontal line in the graph. Therefore, in the illustrated embodiment, the cavitation event starts at about 0.18 ms, and the activity prior to the 0.18 ms mark is considered free of the cavitation events.

FIG. 7C is a schematic view of the cavitation spots in accordance with an embodiment of the present technology. The upper schematics in FIG. 7C indicates the probability of cavitation, and the lower schematics indicates the persistence of cavitation. As explained above, in different embodiments of the inventive technology multiple cavitation spots are generated within the target tissue to promote absorption of the treatment composition.

For the illustrated embodiment, the pulsed focused ultrasound (pFUS) exposures were applied to 20 separate positions within the target sample. Cavitation probability (upper schematics) at each pressure level is defined as the percentage of the positions at which at least one cavitation event was observed. Cavitation persistence (lower schematics) is defined as the percentage of the focused ultrasound pulses that induced a cavitation event among all the pulses delivered within a single treatment position.

At each cavitation spot of the lower schematics K pulses were delivered. If each of the delivered pulses initiates cavitation, the corresponding cavitation persistence would be 100%. However, the 100% cavitation persistence may not be achievable in all cases. For example, although the first pulse (or the first few pulses) may successfully induce cavitation, likely from the pre-existing bubble nuclei, these cavitation bubbles may dissolve before the next pulse arrives, thus depriving these subsequent ultrasound pulses from the appropriate starting nuclei.

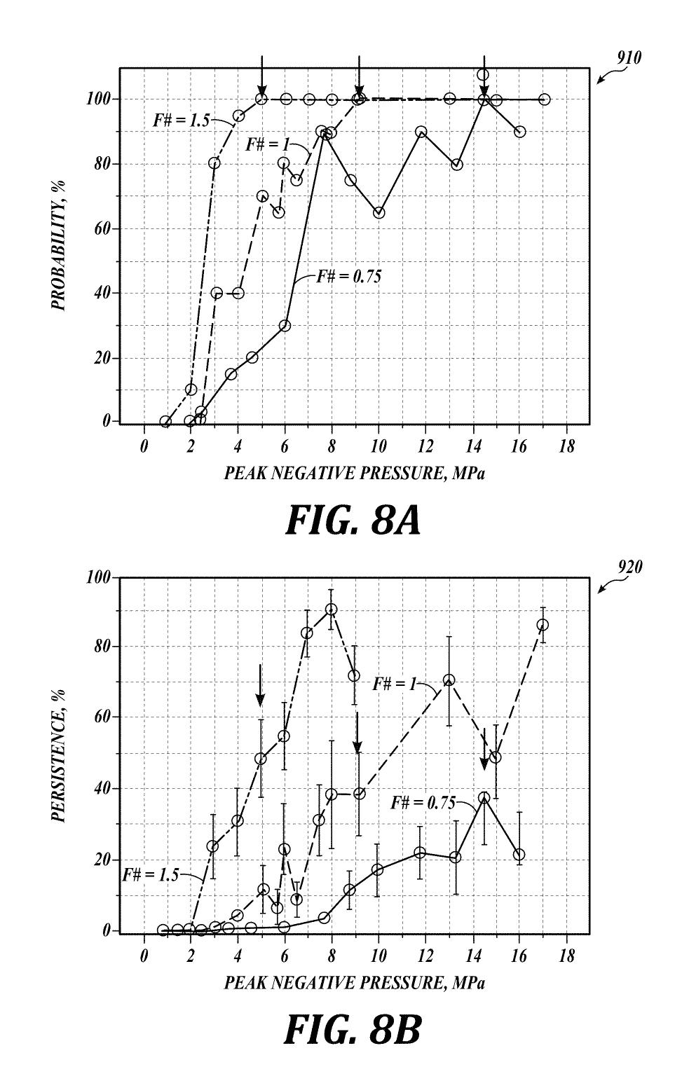

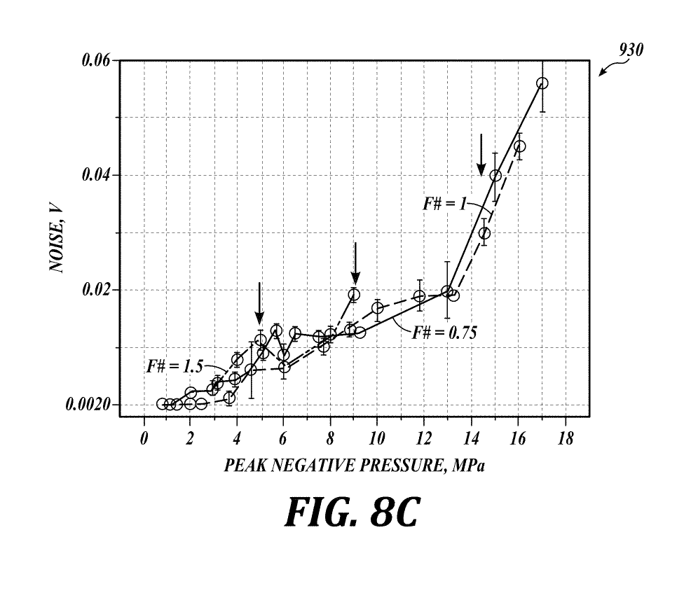

FIGS. 8A and 8B are graphs of probability and persistence, respectively, of the cavitation bubble formation in accordance with embodiments of the present technology. The horizontal axis in FIG. 8A represents the peak negative pressure in MPa and the vertical axis represents the probability of cavitation in percentage. The three groups of data correspond to F-numbers of 1.5, 1, and 0.75. The symbols represent the measurement results and the lines correspond to the simulation results. The arrows that point downward mark the peak negative pressures at which the 100% cavitation probability was achieved. The cavitation probability was calculated over 20 pulsed focused ultrasound locations for each peak negative pressure level. The cavitation probability is different for the transducers for different F-numbers. Generally, cavitation probability of 100% is achieved at the ultrasound output level at which a shock wave forms at the target (i.e., at the focus of the ultrasound). Furthermore, the cavitation probability of 100% is achieved at smaller peak negative pressures for the lenses having larger F-number. The cavitation probability of 100% was reached for lenses with all F-numbers, indicating successful outcome of applying the ultrasound at the target.

FIG. 8B shows the persistence of cavitation for the tested embodiment. Again, the arrows pointing downward mark the peak negative pressures at which the 100% cavitation probability was achieved. The persistence did not reach 100% for any of the transducers. This suggests that, although the first pulse (or the first few pulses) at lower pressure levels successfully induced cavitation, likely from the pre-existing nuclei, these bubbles dissolved before the next pulse arrived so that the subsequently—arrived pulses did not encounter appropriate nuclei. The persistence is markedly different for the transducers with different F-numbers, and was consistently higher for the transducers with higher F-numbers.

FIG. 8C is a graph of ultrasound noise vs. peak negative pressure in accordance with an embodiment of the present technology. The arrows pointing down (numeral 935) mark the peak negative pressures at which the 100% cavitation probability was achieved. In general, the ultrasound emission level detected by the PCD appears to be independent of the transducer F-number. The observable noise in volts ranges from about 0.01 to about 0.04 volts.

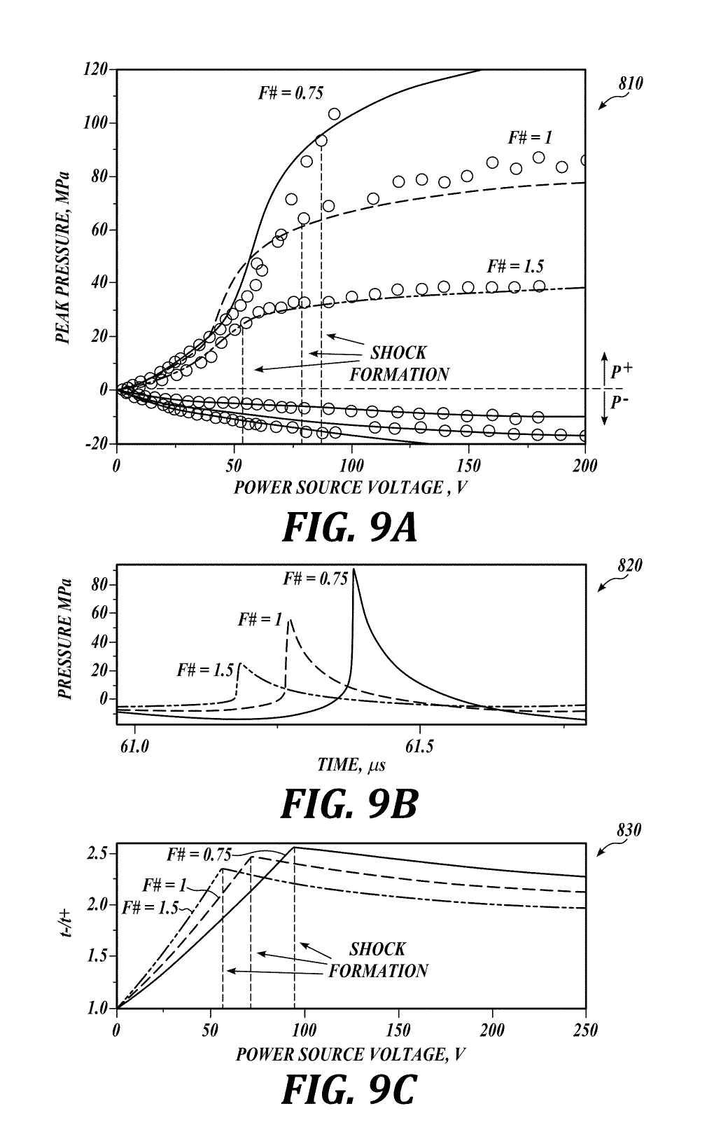

FIGS. 9A-9C are graphs of shock formation in accordance with embodiments of the present technology. The horizontal axis of the graph in FIG. 9A shows the voltage amplitude of the power source. The vertical axis shows the peak positive and peak negative pressures in MPa. The three groups of data correspond to F-numbers of 1.5, 1, and 0.75. The symbols represent the measurement results and the lines correspond to the simulation results. The peak negative pressure was within the (−2.3)-(−5.5) MPa range. The peak positive pressure arranged from about 20 MPa to about 90 MPa at the target area.

FIG. 9B shows one cycle of a periodic pressure waveform generated at the focus of a 1.5-MHz ultrasound source. The horizontal axis of the graph in FIG. 9B shows time in microseconds. The vertical axis shows acoustic pressure in MPa. The three groups of data correspond to F-numbers of 1.5, 1, and 0.75. At the point of shock formation all waveforms were significantly nonlinearly distorted and contained fully develop shock front having a significant peak positive pressure over the corresponding peak negative pressure.

The horizontal axis of the graph in FIG. 9C shows the voltage amplitude of the power source in volts. The vertical axis shows the ratio of the durations of the negative-pressure and positive-pressure portions of the waveform: t⁻/t⁺ (t⁻ being the duration of the negative-pressure portion, or the rarefaction, t⁺ being the duration of the positive-pressure portion, or the compression). The three groups of data correspond to F-numbers of 1.5, 1, and 0.75. The symbols represent the measurement results and the lines correspond to the simulation results. The largest asymmetry of the waveform, corresponding to the greatest ratio of t⁻/t⁺, represents the formation of the fully-developed shocks at the focus, i.e., at the target location.

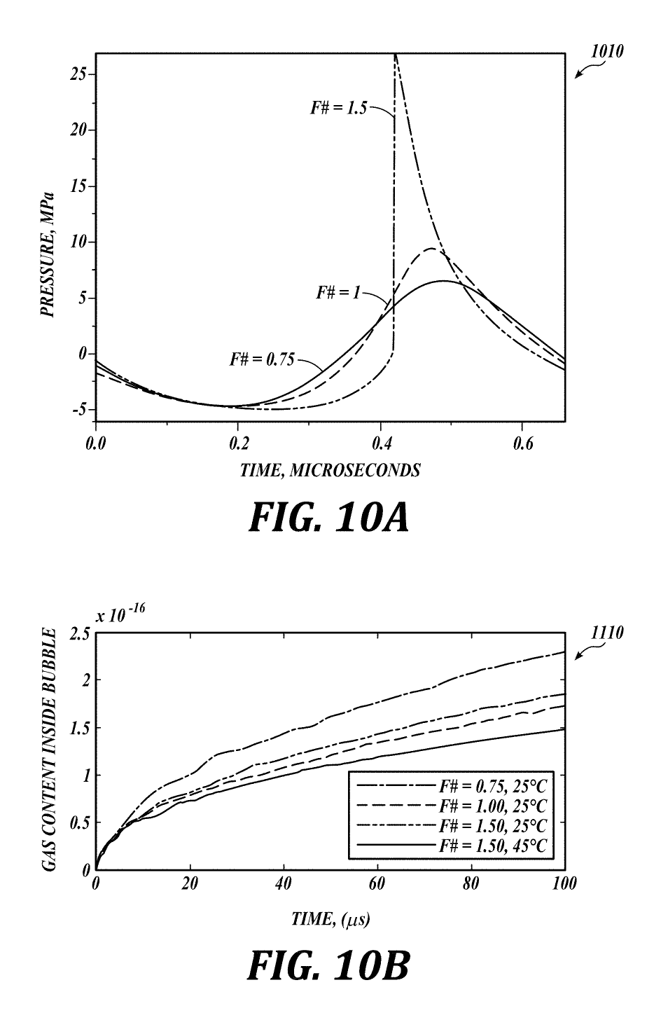

FIGS. 10A and 10B are graphs of pressure waveform and gas content time-history, respectively, of the cavitation bubbles in accordance with embodiments of the present technology. The horizontal axes of the graphs in FIGS. 10A and 10B show the time in microseconds. The vertical axis of the graph on FIG. 10A shows the acoustic pressure at the location of the cavitation bubble in MPa. The vertical axis of the graph on FIG. 10B shows the gas content inside the cavitation bubble in moles.

FIG. 10A shows different levels of nonlinear distortion for different F-numbers. FIG. 10B indicates diffusion of the gas into bubble caused by the excitation waveforms. Although growth occurs for all waveforms, the most rapid growth is caused by the most asymmetrical waveforms with shocks corresponding to F-number 1.5.

Many embodiments of the technology described above may take the form of computer- or controller-executable instructions, including routines executed by a programmable computer or controller. Those skilled in the relevant art will appreciate that the technology can be practiced on computer/controller systems other than those shown and described above. The technology can be embodied in a special-purpose computer, controller or data processor that is specifically programmed, configured or constructed to perform one or more of the computer-executable instructions described above. Accordingly, the terms “computer” and “controller” as generally used herein refer to any data processor and can include Internet appliances and hand-held devices (including palm-top computers, wearable computers, cellular or mobile phones, multi-processor systems, processor-based or programmable consumer electronics, network computers, mini computers and the like).

From the foregoing, it will be appreciated that specific embodiments of the technology have been described herein for purposes of illustration, but that various modifications may be made without deviating from the disclosure. Moreover, while various advantages and features associated with certain embodiments have been described above in the context of those embodiments, other embodiments may also exhibit such advantages and/or features, and not all embodiments need necessarily exhibit such advantages and/or features to fall within the scope of the technology. Accordingly, the disclosure can encompass other embodiments not expressly shown or described herein.

I/we claim:

1. A method for delivering a treatment composition to a target tissue using ultrasound, the method comprising:
directing ultrasound waveforms toward a target tissue of a patient;
generating ultrasound waveforms with shock fronts at the target tissue of a patient;
generating cavitation inside the target tissue of a patient by the ultrasound with shock fronts; and
within a time period of +/−1 week from generating the cavitation, delivering the treatment composition to the patient, wherein an absorption of the treatment composition by the target tissue is increased by the cavitation inside the target tissue.

2. The method of claim 1, wherein the time period ranges from −1 hour to +48 hours.

3. The method of claim 1, wherein the ultrasound waveform is produced by an ultrasound transducer having an F-number within a 1-5 range.

4. The method of claim 3, wherein a characteristic dimension of the ultrasound transducer is less than 8 cm.

5. The method of claim 1, wherein producing the ultrasound waveforms comprises:
producing a first burst of ultrasound waveforms within a first period of time, wherein the first period of time is shorter than 1 ms, and wherein the first burst of ultrasound waveforms is focused at a first segment of the target tissue; and
producing a second burst of ultrasound waveforms within a second period of time, wherein the second period of time is shorter than 1 ms, wherein the second burst of ultrasound waveforms is focused at a second segment of the target tissue, and wherein the second segment is different than the first segment.

6. The method of claim 5, wherein adjacent bursts of the ultrasound waveforms are separated by a rest time, wherein a ratio of a duration of the bursts and a duration of the rest times is a duty cycle of the treatment, and wherein the duty cycle of the treatment is less than 1%.

7. The method of claim 5, wherein a frequency of the ultrasound waveforms within the first burst and the second burst ranges from 0.5 MHz to 3 MHz.

8. The method of claim 5, wherein a burst-to-burst frequency is 1-200 Hz.

9. The method of claim 1, wherein the ultrasound shock fronts inside the target tissue have a peak negative pressure within a range of −2 MPa to −10 MPa, and a peak positive pressure within a range of 10 MPa to 70 MPa.

10. The method of claim 1, wherein the treatment composition comprises a chemotherapy treatment composition.

11. The method of claim 1, wherein the treatment composition comprises a gene therapy.

12. The method of claim 1, wherein the target tissue comprises a tumor.

13. The method of claim 1, wherein the treatment composition is administered before generating the cavitation, but not after generating the cavitation.

14. The method of claim 1, wherein the treatment composition is administered after generating the cavitation, but not before generating cavitation.

15. A system for delivering a treatment composition to a target tissue using ultrasound, the system comprising:
an ultrasound transducer configured for directing ultrasound waveforms toward a target tissue of a patient, wherein the nonlinear propagation effects generate ultrasound shock fronts at the target tissue of a patient, and wherein the ultrasound shock fronts generate cavitation inside the target tissue; and
the treatment composition delivered within a time period of +/−1 week from generating the cavitation, wherein an absorption of the treatment composition by the target tissue is increased by the cavitation inside the target tissue.

16. The system of claim 15, further comprising a lens attached to the ultrasound transducer, wherein the lens has an F-number within a 1-5 range.

17. The system of claim 15, wherein a characteristic dimension of the ultrasound transducer is less than 8 cm.

18. The system of claim 15, wherein a frequency of the ultrasound waveforms within the first burst and the second burst ranges from 0.5 MHz to 3 MHz.

19. The system of claim 15, wherein a burst-to-burst frequency is 1-200 Hz.

20. The system of claim 15, wherein the ultrasound shock fronts inside the target tissue have a peak negative pressure within a range of −2 MPa to −10 MPa, and a peak positive pressure within a range of 10 MPa to 70 MPa.
